(19) United States
(12) Patent Application Publication
(10) Pub. No.: US 2014/0303119 A1
(43) Pub. Date: Oct. 9, 2014

(54) METHODS, APPARATUS, AND COMPOUNDS FOR THE TREATMENT OF CENTRAL RETINAL VEIN OCCLUSION AND OTHER CONDITIONS

(71) Applicant: Daniel John DiLorenzo, Chicago, IL (US)

(72) Inventor: Daniel John DiLorenzo, Chicago, IL (US)

(73) Assignee: DiLorenzo Biomedical, LLC, Houston, TX (US)

(21) Appl. No.: 13/798,049

(22) Filed: Mar. 12, 2013

(51) Int. Cl. A61K 31/737; A61K 31/4365; A61K 31/122; A61K 31/727; A61K 31/37

(52) U.S. Cl. CPC A61K 31/737; A61K 31/727; A61K 31/37; A61K 31/122; A61K 31/4365
USPC 514/56; 514/54; 514/457; 514/681; 514/301

(57) ABSTRACT

The present invention teaches a method and new use for anticoagulant compounds for the treatment of central retinal vein occlusion (CRVO) and branch retinal vein occlusion (BRVO) and other conditions. CRVO and BRVO represent debilitating conditions for which there is currently no effective therapy. The present invention teaches a new therapy, including new uses for existing pharmacological agents, comprising enoxaparin sodium, warfarin, clopidrogel, and others, in the treatment of these and other conditions.

### RELATED APPLICATIONS

All patents, patent applications, patent publications, and non-patent publications listed are hereby incorporated by reference.

This application incorporates by reference all patents listing as inventor or co-inventor Daniel J. DiLorenzo.

This application is a continuation of and incorporates by reference U.S. Patent Application Ser. No. 61/335,917 (Docket CRVO 01.01p1), entitled “METHODS AND COMPOUNDS FOR THE TREATMENT OF CENTRAL RETINAL VEIN OCCLUSION AND OTHER THROMBOTIC AND EMBOLIC CONDITIONS”, filed Jan. 13, 2010, which names as inventor Daniel John DiLorenzo, and which is incorporated by reference, which is a continuation of and claims the benefit of US Provisional Patent Application Filed May 26, 2009 and which was properly mailed (in Express Mail Label # EH 505759105 US) which was received, damaged in transit by the US Postal Service, and presented for delivery to USPTO; however it was refused and returned because of damage in transit, the documentation for which was submitted in the above cited application which was subsequently filed and received.

### BACKGROUND OF THE INVENTION

1. Field of the Invention

The present invention relates generally to treatment of thrombotic disease and, more particularly, to thrombotic disease of the retina, including but not limited to central retinal vein occlusion (CRVO), branch retinal vein occlusion (BRVO), arterial occlusive disease of the retina, and other diseases involving the retina, nervous system, deep and superficial veins including those of the extremities (legs and arms), torso, abdomen, spinal cord, and brain, vessels of other organ systems, and other components of various organ systems.

Central retinal vein occlusion is one of the most common causes of visual loss, and its prevalence has been estimated to be between 0.1% and 0.4% in individuals over age 40 [1,2]. The condition was first recognized in 1855; however, no effective treatment has been identified, and therapeutic approaches and clinical management remains controversial. Venous occlusion in CRVO causes persistent macular edema, macular ischemia, and neovascular glaucoma, each of which contribute to the visual loss and blindness suffered by patients afflicted with CRVO.

2. Related Art

Clinical and physiologic studies have demonstrated the ineffectiveness of therapies attempted to date. Management of CRVO, once diagnosed, typically involves observation and subsequent treatment of neovascularization.

A variety of therapies have been tried for CRVO. These include medical therapy with troxerutin, a derivative of the naturally occurring bioflavonoid rutin, which improves erythrocyte deformability; this has been shown to provide an improvement in visual acuity, retinal circulation time, and in macular edema versus placebo (Glacet-Bernard A, Coscas G, Chabanel A, et al. A randomized, double-masked study on the treatment of retinal vein occlusion with troxerutin. Am J Ophthalmol 1994; 118:421-9.1.

Ticlodipine, an agent which inhibits platelet aggregation, has shown some benefit, with one randomized clinical study demonstrating a trend for improvement in the treatment group versus placebo, but no statistical significance was achieved (Houtsmuller A J, Vermeulen J A, Klompe M, et al. The influence of ticlopidine on the natural course of retinal vein occlusion. Agents Actions Suppl 1984; 15:219-29.).

Pentoxifylline, an oral agent used in the treatment of peripheral vascular disease, improves perfusion of occluded vessels by increasing red blood cell deformability, reducing blood viscosity, and decreasing potential for platelet aggregation. A small randomized clinical study showed retinal flow velocity in patients treated with pentoxifylline versus placebo at 4 weeks (De Sanctis M T, Cesarone M R, Belcaro G, et al. Treatment of retinal vein thrombosis with pentoxifylline: a controlled, randomized trial. Angiology 2002; 53(suppl):S35-8).

Prostacyclin given intravenously, was evaluated in a small randomized clinical study; and no significant difference in visual acuity or in progression to neovascularization was found versus placebo (Zygulska-Mach H, Mirkiewicz-Sieradzka B, Kostka-Trabka E, et al. Evaluation of the effectiveness of prostacyclin in the treatment of thrombosis of the central retinal vein using a double-blind method [in Polish]. Klin Oczna 1992; 94:13-5).

Streptokinase, a fibrolytic agent, was evaluated in a randomized clinical trial comparing continuous infusion followed by warfarin with no specific treatment. Only 5 of 20 in the treated group versus 12 of 20 in control group showed deterioration in vision; however, 3 of 20 of the treated group experienced massive vitreous hemorrhage within 72 hours with persistence of blindness in those eyes (Kohner E M, Hamilton A M, Bulpitt C J, Dollery C T. Streptokinase in the treatment of central retinal vein occlusion: a controlled trial. Trans Ophthalmol Soc U K 1974; 94:599-603; Kohner E M, Pettit J E, Hamilton A M, et al. Streptokinase in central retinal vein occlusion: a controlled clinical trial. Br Med J 1976; 1:550-3.).

Tissue plasminogen activator (tPA) was evaluated in a prospective nonrandomized study involving in 96 patients with CRVO; 1 patient suffered a fatal stroke, and 3 patients developed severe intraocular bleeding (Elman M J. Thrombolytic therapy for central retinal vein occlusion: results of a pilot study. Trans nm Ophthalmol Soc 1996; 94:471-504).

Because of the limited efficacy and the significant risk of adverse events including disastrous intraocular hemorrhage, stroke, and fatality, fibrinolytic therapy is generally not used, and conservative management involving observation and subsequent treatment of neovascularization is the standard of care.

Hemodilution involves the administration of fluids for the dilution of blood, particularly that of red cells. The rationale for this therapy has been based upon the observation of abnormal red cell deformability, increased plasma viscosity, and increased hematocrit and fibrinogen levels in some patients with CRVO. As in the treatment of cerebral vasospasm, reducing hematocrit to lower plasma viscosity is believed to improve retinal microcirculation and perfusion (Mohamed Q, Interventions for Central Retinal Vein Occlusion: An Evidence-Based Systematic Review, Ophthalmology 2007; 114:507-519). Results with this therapy are mixed, as several studies have show improvement in visual acuity, though some have shown decline or no benefit.

Corticosteroids administered intravitreally, such as intravitreal triamcinolone, for the treatment of macular edema has been reported in case reports and small series, typically uncontrolled and retrospective. These therapies tend to be short-lived and require repeat injections to maintain improvement. The risks are significant and include sterile and infectious endophthalmitis, vitreous hemorrhage, retinal detachment, raised intraocular pressure occasionally requiring surgery, and cataract formation Mohamed Q, Interventions for Central Retinal Vein Occlusion: An Evidence-Based Systematic Review, Ophthalmology 2007; 114:507-519).

Laser treatments, such as panretinal photocoagulation, has been shown in some studies to provide a reduced incidence of neovascular glaucoma; however, not surprisingly, the adverse effect of a reduction in visual fields as tested with Goldmann perimetry has been documented.

Additional treatments advocated include fibrinolytics, corticosteroids, acetazolamide, and isovolemic hemodilution. More recently, agents which inhibit vascular proliferation have been tried intravitreally to improve efficacy and limit systemic side effects. Lasers have been used to deliver therapy by a variety of mechanisms, including panretinal photocoagulation, grid pattern photocoagulation, and chorioretinal anastomosis. Surgical approaches have included vitrectomy, surgically induced chorioretinal anastomosis, direct venous cannulation with injection of fibrinolytics, and radial optic neurotomy (RON).

Although varying levels of benefit have been reported for some of these, none has demonstrated clear efficacy in treating this condition. As a result, controversy in management persists, and a variety of more invasive and risky procedures are being attempted in a desperate attempt to treat this potentially devastating condition.

The current state is well captured by current publications and clinical media cited below. Examples from published literature in peer-reviewed journals, publicly accessible clinical reference websites, and clinical centers are provided to illustrate the dire need for an effective therapy for this disabling condition.

Peer Reviewed Literature:

Peer reviewed literature in the leading journals attests to the absence of medical treatments and advocates against the use of anticoagulants. In a recent review of CRVO, Mohamed et al state “There is limited evidence that any oral or systemic anticoagulation or rheological agent can significantly affect the outcome of CRVO. Although troxerutin and ticlodipine showed a trend for improvement, the evidence supporting these modalities was limited14,15 (level B, III). Ticlodipine has uncommon (0.5%-3%) but very serious hematological toxicity, including neutropenia, thrombocytopenia, and aplastic anemia requiring regular monitoring. Clopidrogel, a newer agent with an almost identical structure and mechanism of action, increasingly is used preferentially due to a reduced incidence of blood disorders and no need for regular monitoring. No randomized study has assessed the role of clopidrogel in retinal venous thrombosis, and routine use of these agents is not recommended for CRVO.” (Mohamed Q, et al., “Interventions for Central Retinal Vein Occlusion An Evidence-Based Systematic Review”, American Academy of Ophthalmology, volume 114, pages 507-519; quotation is from treatment section on p. 513)

Clinical Reference Websites:

Current material from well respected clinical reference websites supports this absence of medical therapy for CRVO.

The eMedicine clinical reference website article entitled “Central Retinal Vein Occlusion” by Lakshmana M Kooragayala, M D, Vitreo-retinal Surgeon, Marietta Eye Clinic and Updated: Dec. 11, 2008 states. “No known effective medical treatment is available for either the prevention of or the treatment of central retinal vein occlusion (CRVO). Identifying and treating any systemic medical problems to reduce further complications is important. Because the exact pathogenesis of the CRVO is not known, various medical modalities of treatment have been advocated by multiple authors with varying success in preventing complications and in preserving vision.” (under eMedicine Specialties>Ophthalmology>Retina; at URL: http://emedicine.medscape.com/article/1223746-print, accessed May 24, 2009)

Another article published on the eMedicine website entitled “Retinal Vein Occlusion” by Mark Fonrose, M D, FACEP, Assistant Professor of Emergency Medicine, Kings County Hospital Center/State University of New York and updated: Aug. 25, 2008 states, “Treatment remains controversial at the present time. The Canadian Journal of Ophthalmology 2008 noted “No intervention has emerged as the standard of care. Current management in most centers is close observation for complications and treatment as they arise.” Another 2008 study notes that many different interventions have been advocated, but evidence is lacking as to their merit. Several randomized clinical trials are underway at the present time.” (from eMedicine Specialties>Emergency Medicine>Ophthalmology, http://emedicine.medscape.com/article/798583-print, accessed May 24, 2009)

The Merck Manual clinical reference website article entitled “Central Retinal Vein Occlusion” revised December 2008 by Sunir J. Garg, M D states, “There is no generally accepted medical therapy for occlusion itself. However, if neovascularization develops, panretinal photocoagulation should be initiated because it may decrease vitreous hemorrhages and prevent neovascular glaucoma. Clinical trials are investigating intravitreal injection of corticosteroids and anti-vascular endothelial growth factor drugs.” (from http://www.merck.com/mmpe/sec09/ch106/ch106d.html, accessed May 24, 2009).

Clinical Patient Informational and Advertising Media:

Clinical informational and advertising media, such as that currently published on academic institution and private clinic websites, further supports the current absence of effective medical therapy for CRVO.

A case published on the Harvard/Massachusetts Eye and Ear Infirmary (MEEI) website entitled “43 year old man with 3 days of blurry vision OS” by Nisha Acharya, M. D. concurs, “There is no proven benefit of aspirin, steroids, or systemic anticoagulation in the treatment of CRVO. Patients are monitored for the development of iris neovascularization, and pan-retinal photocoagulation is performed if it develops. Underlying systemic conditions should be treated (hypertension/diabetes). Additionally, CRVO in young patients should prompt an evaluation for a hypercoagulable state.” (from Digital Journal of Ophthalmology 2002, Volume 8, Number 7, on http://www.djo.harvard.edu/print.php?url=/physicians/gr/295&print=1, accessed May 24, 2009)

A large nonprofit multi-site multi-specialty healthcare delivery organization in Boston states on its website, “Unfortunately, central retinal vein occlusion is a very frustrating problem and final vision is often not very good. The visual outcome is not improved by any treatment since there is no way to alter the natural progression of the vein occlusion itself.” (Harvard Vanguard Medical Associates, http://www.harvardvanguard.org/eye/VScrvo2.asp, accessed May 24, 2009)

A clinical website from a New York vitreoretinal subspecialty practice states: “Presently there is no proven treatment for CRVO. When CRVO causes complications such as abnormal new blood vessel growth, laser treatment to reverse the growth of new blood vessels is often required. If untreated, these abnormal blood vessels can cause vitreous hemorrhage and glaucoma. Intravitreal medications may be recommended in some cases to cause regression of new blood vessels and resolution of macular edema” (Mohawk Valley Retina, New Hartford, N.Y., http://www.mvretina.com/education/13.html, accessed May 24, 2009).

Additional private clinic websites support this unfortunate state of affairs, “In summary, retinal vein occlusions develop from obstruction of the venous outflow from the eye. The blockage may vary in size and location, accounting for a wide range of retinal outcomes. Some of the complications of retinal vein occlusion may be appropriately managed with laser treatment. It is hoped that through further research, even better strategies for prevention and management will be developed.” (The Angeles Vision Clinic, Port Angeles, Wash., http://www.avclinic.com/retinal_vein_occlusion.htm, accessed May 24, 2009)

Conclusion:

There remains a dire need for an effective therapy for CRVO, BRVO, and other related and thrombotic conditions. The present invention, comprising a new method of use and apparatus for delivery, offers a solution to this need and offers the potential to prevent millions of cases of visual loss and blindness.

### SUMMARY OF THE INVENTION

The present invention teaches apparatus and methods for treating a multiplicity of diseases, including central retinal vein occlusion (CRVO), and other diseases. There disease or conditions include but are not limited to branch retinal vein occlusion (BRVO), deep vein thrombosis (DVT), superficial vein thrombosis, cavernous sinus thrombosis, superior sagittal sinus thrombosis, pulmonary embolism (PE), and other thrombotic, embolic, thromboembolic conditions, and arterial and venous occlusive conditions. The invention taught herein employs a novel therapy for the treatment of central retinal vein occlusion (CRVO) or other occlusive conditions.

The present invention teaches the use of acute, subacute, and chronic anticoagulation in the acute treatment and long term management of CRVO. Immediately upon diagnosis of CRVO, a patient is administered anticoagulation, preferably with an immediate or short latency of onset, such as intravenous heparin or subcutaneous enoxaparin sodium, or equivalent agent, or agent with similar or equivalent mechanism or effect. This therapy is continued until the CRVO is found to resolve, typically for a duration of at least 3 months. This may then be continued for a longer duration, such as 6 months, 9 months, 1 year, 2 years, other duration, permanently, or indefinitely. Alternatively, this therapy may be tapered down in frequency and/or in dose. Furthermore, another form of therapy may be phased in, including but not limited to warfarin or other agent.

Effective dosing for this therapy includes but is not limited to 1 mg/kg q12 hours. Higher dosing levels, including but not limited to >1 mg/kg, >1.5 mg/kg, >2 mg/kg, >3 mg/kg, >4 mg/kg, >5 mg/kg, >6 mg/kg, >7 mg/kg, >8 mg/kg, >9 mg/kg, >10 mg/kg, and other doses may be used without departing from the present invention. Lower dosing levels, including but not limited to <1 mg/kg, <0.5 mg/kg, <0.25 mg/kg, <0.125 mg/kg, <0.1 mg/kg, <0.075 mg/kg, <0.05 mg/kg, <0.025 mg/kg, <0.125 mg/kg, <0.01 mg/kg, <0.0075 mg/kg, <0.005 mg/kg, <0.0025 mg/kg, <0.00125 mg/kg, <0.001 mg/kg, and other doses may be used without departing from the present invention. These and other doses may be given using acute intravenous or subcutaneous routes as well as via chronic routes including but not limited to PICC lines (peripheral indwelling central catheter), central lines, peripheral lines, Hickman catheters, portacath devices, or other routes.

Prior to, during, and following therapy, the patient should be followed with preferably regular, or periodic, or intermittent follow up by a healthcare professional, preferably a retinal specialist ophthalmologist. Follow up visits preferably include slit lamp evaluation, tonometry (measurement of intraocular pressure) or equivalent study, retinal fluorescein imaging to assess retinal vascular flow. Once CRVO or BRVO has resolved, the therapy may be changed or discontinued, depending on preference of the physician and patient. This may be influenced by the diagnosis of and available treatments for any underlying coagulopathy or vascular abnormality.

The preferred treatment regimen is enoxaparin to be administered immediately upon diagnosis at therapeutic levels, 1 mg/kg subcutaneously every 12 hours and to continue until retinal examination indicates resolution of thrombosis. Following this phase, dosing may be decreased and or discontinued. Depending on risk factors fro recurrence, enoxaparin or another agent may be continued to serve a prophylactic or preventative role. One preferred agent to serve a preventative role is clopidogrel (Brand name: Plavix; systematic IUOAC name: (+)-(S)-methyl 2-(2-chlorophenyl)-2-(6,7-dihydrothieno[3,2-c]pyridin-5(4H)-yl)acetate)

This preferred treatment is novel and represents a significant advance over the current therapies available, most of which address the secondary effects such as neovascularization, and elevated intraocular pressure. The use of other anticoagulant agents may also be used singly or in combination, and these are included without departing from the present invention. Currently, approximately ⅓ of patients experience some improvement in vision, ⅓ stabilize with a permanent deficit, and ⅓ progress to blindness in the involved eye. Occasionally, in patients in whom blindness develops, complications including intolerable pain, require enucleation of the eye, a potentially disfiguring and certainly psychologically devastating event. The situation for affected patients is dire, and a clear need for a better treatment exists.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

Furthermore all patents claiming priority to US Provisional applications, US patent applications, US patents, and International Patents and patent applications naming Daniel DiLorenzo as an inventor or co-inventor are included by reference.

U.S. Pat. No. 5,389,618, filed Jul. 16, 1993 and issued Feb. 14, 1995, and all related US and international applications and patents are included by reference. U.S. Pat. No. RE 38,743 E, a Reissue of U.S. Pat. No. 5,389,618, which was filed Feb. 14, 2005 and issued Jun. 14, 2005, and all related US and international applications and patents are included by reference.

### BRIEF DESCRIPTION OF DRAWINGS

FIG. 1 depicts fundoscopic images of the human retina in its normal state and in states of central retinal venous occlusive (CRVO) disease. A: Normal Retina, B: Central Retinal Vein Occlusion (CRVO), C: Central Retinal Vein Occlusion (CRVO).

FIG. 2 depicts images of the human retina in states of venous occlusive disease, allowing comparison of branch retinal venous occlusion (BRVO) and central retinal venous occlusion (CRVO). A: Branch Retinal Vein Occlusion, B: Branch Retinal Vein Occlusion, C: Central Retinal Vein Occlusion, D: Central Retinal Vein Occlusion.

FIG. 3 depicts Fluorescein angiogram images of a normal human retina and a retina with central retinal venous occlusion (CRVO).

FIG. 4 depicts images of the human retina in states of branch retinal venous occlusion (BRVO). A: Fluorescein angiogram at 15 seconds in BRVO, B: Fundoscopic image in BRVO, C: Fluorescein angiogram at 3 minute 13 seconds in BRVO, D: Fluorescein angiogram at 53 seconds in BRVO.

FIG. 5 depicts images of the human retina in states of central retinal venous occlusion (CRVO). A: Fluorescein angiogram at 36 seconds in CRVO, B: Fundoscopic image in CRVO, C: Fluorescein angiogram at 3 minute 58 seconds in CRVO, D: Fluorescein angiogram at 1 minute 16 seconds in CRVO.

FIG. 6 depicts the chemical structure of enoxaparin sodium, a compound for novel therapeutic use taught in the present invention:

FIG. 7 is an outline of current antithrombotic compounds, including Anticoagulants, Antiplatelet Drugs, and Thrombolytic Drugs:

### DETAILED DESCRIPTION OF THE INVENTION

The present invention encompasses a multimodality technique, method, and apparatus for the treatment of several diseases, including but not limited to DISEASE_ENUMERATION.

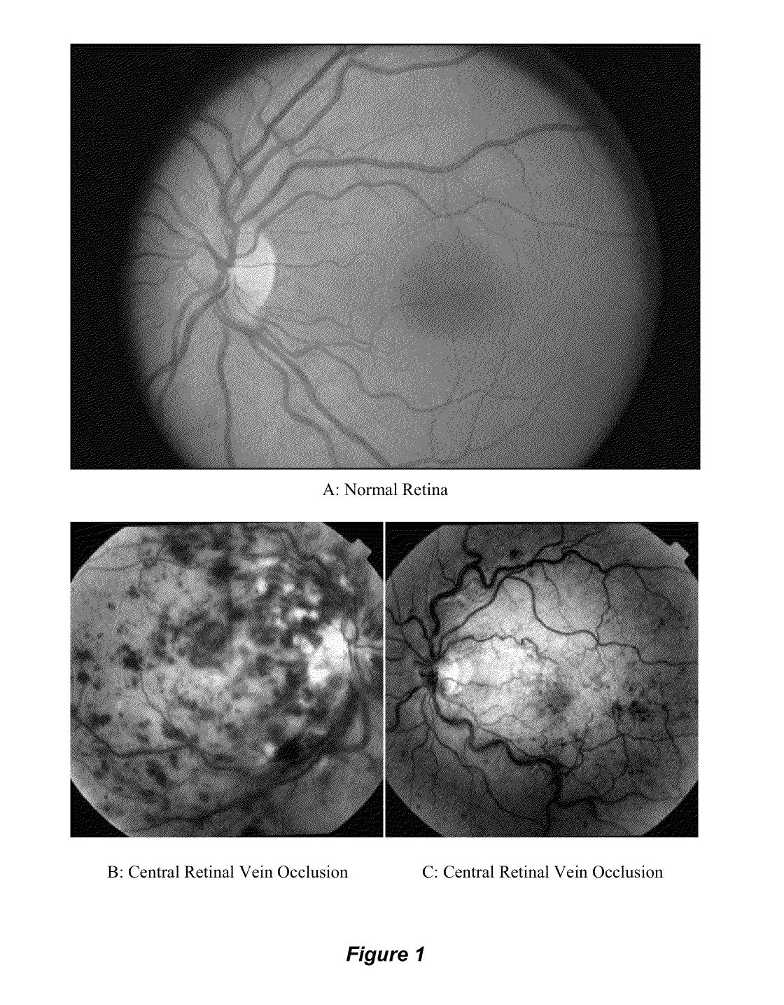

FIG. 1 depicts fundoscopic images of the human retina in its normal state and in states of central retinal venous occlusive (CRVO) disease. A: Normal Retina, B: Central Retinal Vein Occlusion (CRVO), C: Central Retinal Vein Occlusion (CRVO). In the normal human retina shown in FIG. 1A, the central retinal artery and vein enter the globe, divide into branch retinal arteries and veins, respectively, and perfuse the retina. The normal optic disc, a yellow structure with a crisp border is seen on the left. The macula, with the highest density of photoreceptors, is seen slightly to the right of the center of the image. In the human retina with CRVO shown in FIGS. 1B and 1C, delayed or incomplete filling of the retinal vein is seen, and diffuse areas of venous hemorrhage is seen throughout the retina.

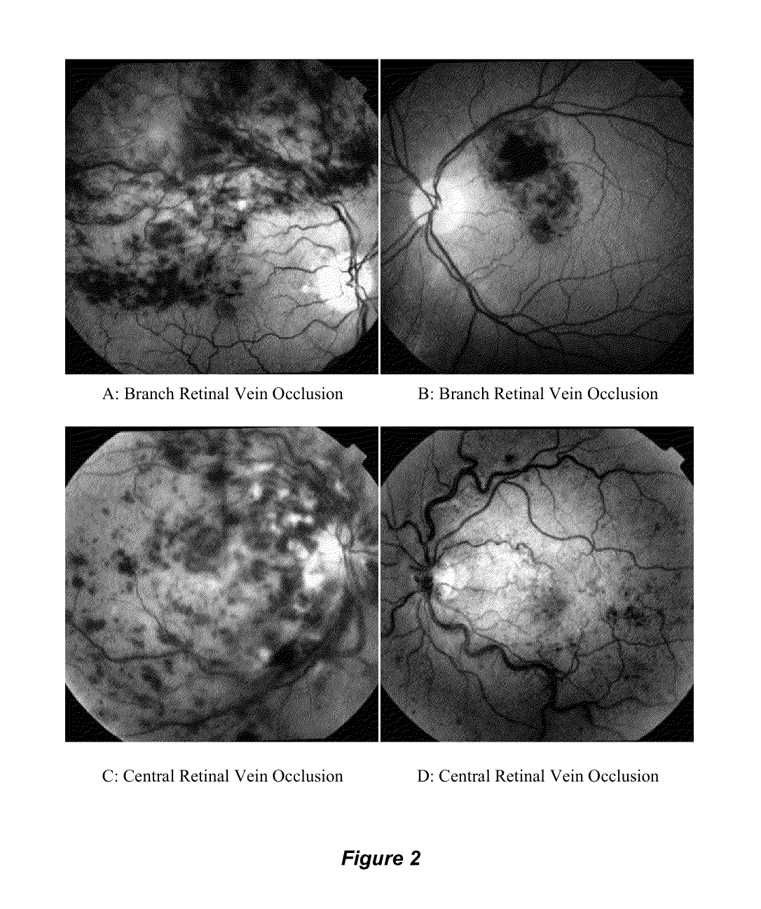

FIG. 2 depicts images of the human retina in states of venous occlusive disease, allowing comparison of branch retinal venous occlusion (BRVO) and central retinal venous occlusion (CRVO). In FIGS. 2A and 2B, Branch Retinal Vein Occlusion (BRVO) is shown, characterized by profound involvement of distinct regions of the retina in which venous drainage is impaired by the occlusion of the respective central retinal vein branch. In FIGS. 2C and 2D (the same as FIGS. 1B and 1C), Central Retinal Vein Occlusion (CRVO) is shown, characterized by profound involvement of the entire retina in which venous drainage is impaired by the occlusion of the central retinal vein.

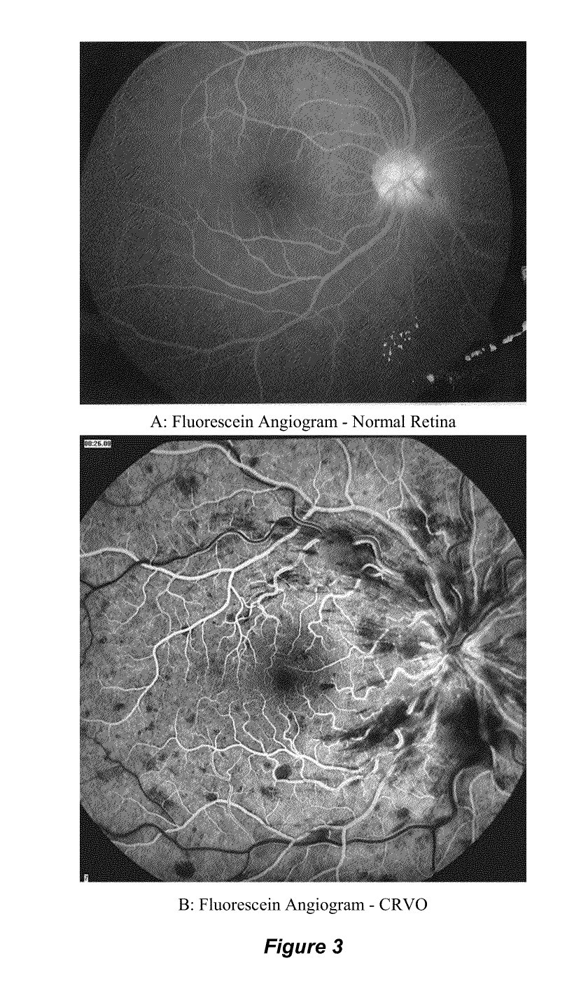

In FIG. 3, Fluorescein angiogram images of a normal human retina (FIG. 3A) and a retina with central retinal venous occlusion (CRVO) (FIG. 3B) are shown. In FIG. 3B, the delayed and impaired filling of the retinal veins and the diffuse retinal venous hemorrhage are seen.

Fluorescein angiography is well known in the field of ophthalmology. Briefly, baseline color and black and white red-free filtered images are taken prior to injection. The black and white images are filtered red-free (with a green filter) to increase contrast, often providing a better image of the fundus than a color image. An bolus of 2-5 cc of sodium fluorescein is injected into a vein in the arm or hand over 6 second interval. A series of black-and-white or digital photographs are taken of the retina before and after the fluorescein reaches the retinal circulation (approximately 10 seconds after injection). The early images allow for the recognition of autofluorescence of the retinal tissues. Photos are taken approximately once every second for about 20 seconds and subsequently less often. A delayed image is obtained at 5 and 10 minutes and occasionally at 15 minutes. A filter is placed in the camera so only the fluorescent, yellow-green light (530 nm) is recorded. Various component of the retinal vascular circulation are assessed during the transit of fluorescein; these are outlined as follows:

0 seconds—injection of fluorescein

9.5 sec—posterior ciliary arteries

10 sec—choroidal flush (or “pre-arterial phase”)

10-12 sec—retinal arterial stage

13 sec—capillary transition stage

14-15 sec—early venous stage (or “lamellar stage”, “arterial-venous stage”)

16-17 sec—venous stage

18-20 sec—late venous stage

5 minutes—late staining

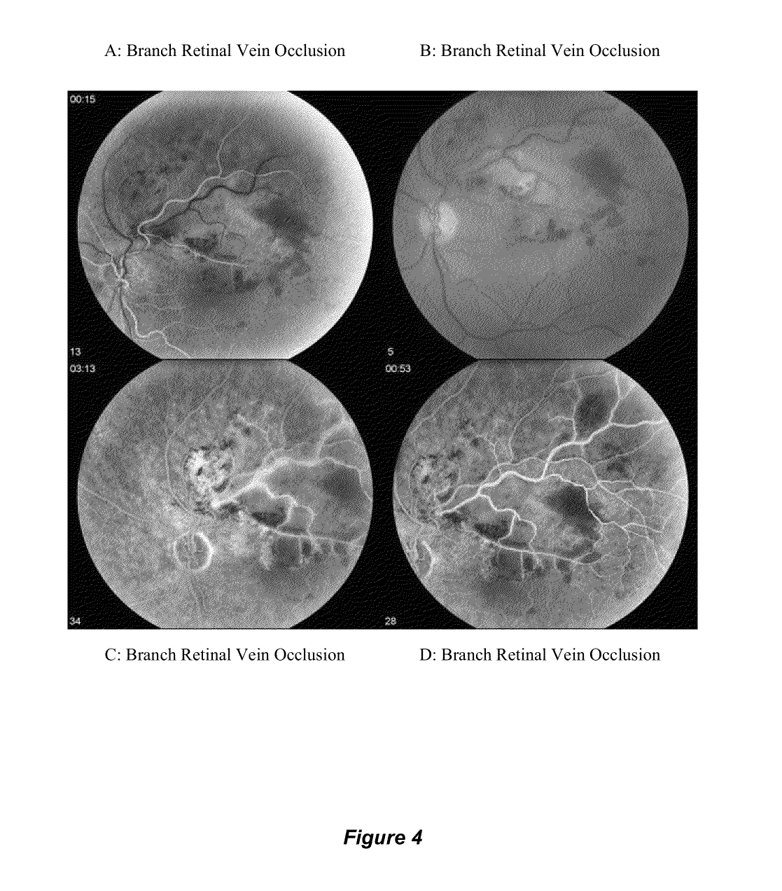

FIG. 4 depicts images of the human retina in states of branch retinal venous occlusion (BRVO). A: Fluorescein angiogram at 15 seconds in BRVO, B: Fundoscopic image in BRVO, C: Fluorescein angiogram at 3 minute 13 seconds in BRVO, D: Fluorescein angiogram at 53 seconds in BRVO. These images show absence of flow in branch retinal veins in the involved branch retinal vein segments and venous hemorrhages in these respective regions, resulting from inadequate venous drainage.

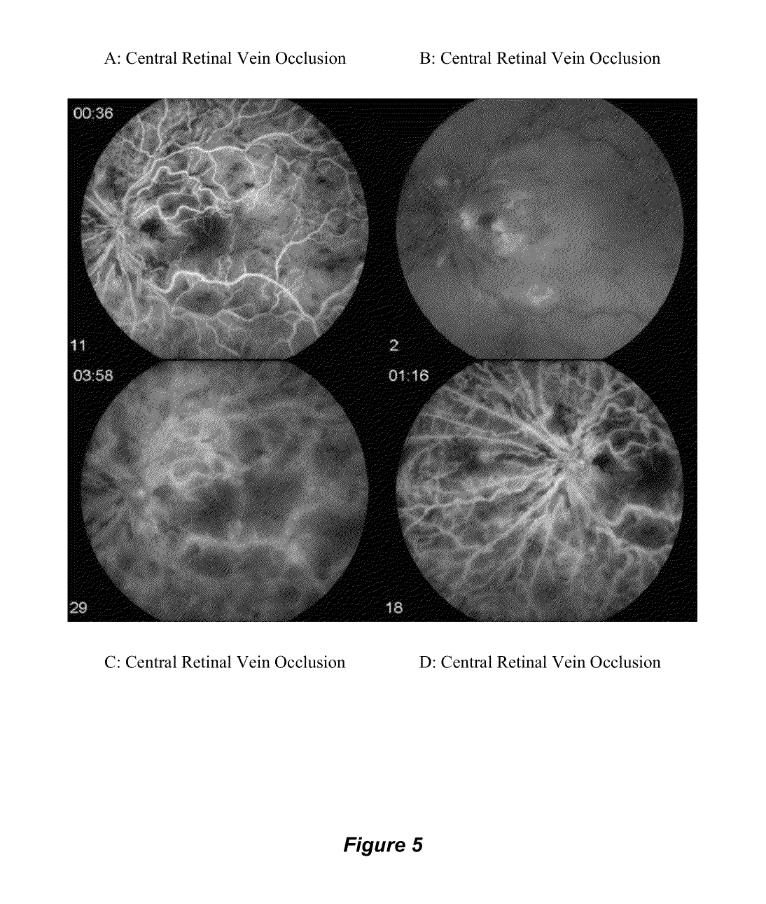

FIG. 5 depicts images of the human retina in states of central retinal venous occlusion (CRVO). A: Fluorescein angiogram at 36 seconds in CRVO, B: Fundoscopic image in CRVO, C: Fluorescein angiogram at 3 minute 58 seconds in CRVO, D: Fluorescein angiogram at 1 minute 16 seconds in CRVO. These images show delayed or absent venous flow throughout entire retina, supplied by the central retinal vein, and diffuse venous hemorrhages, resulting from inadequate venous drainage.

These images attest to the profound retinal damage commonly encountered in these venous occlusive conditions.

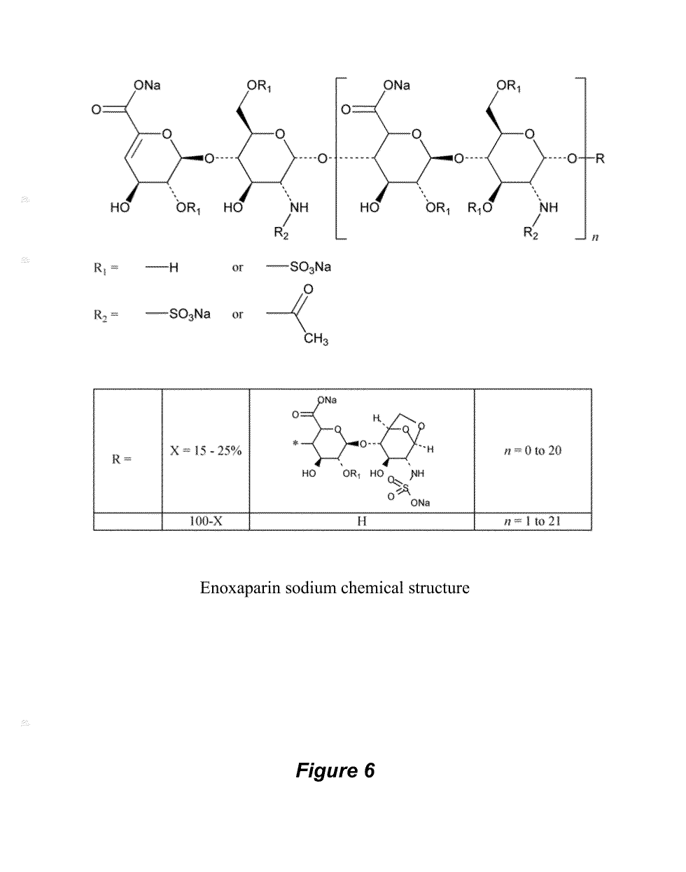

FIG. 6 depicts the chemical structure of enoxaparin sodium, a compound for novel therapeutic use taught in the present invention. The chemical formula is C₂₃H₂₇N. The uses for this compound include (source: Drugs.com, http://www.drugs.com/ingredient/enoxaparin.html#, accessed May 24, 2009):

Acute Coronary Syndrome

Angina

Deep Vein Thrombosis (DVT)

Deep Vein Thrombosis Prophylaxis after Abdominal Surgery

Deep Vein Thrombosis Prophylaxis after Hip Replacement Surgery

Deep Vein Thrombosis Prophylaxis after Knee Replacement Surgery

Deep Vein Thrombosis, Prophylaxis

Heart Attack

The indications for Lovenox, low molecular weight heparin [LMWH] and a brand of enoxaparin sodium, are:
(i) Prophylaxis of deep vein thrombosis (DVT) in abdominal surgery, hip replacement surgery, knee replacement surgery, or medical patients with severely restricted mobility during acute illness (1.1)
(ii) Inpatient treatment of acute DVT with or without pulmonary embolism (1.2)
(iii) Outpatient treatment of acute DVT without pulmonary embolism. (1.2)
(iv) Prophylaxis of ischemic complications of unstable angina and non-Q-wave myocardial infarction [MI] (1.3)
(v) Treatment of acute ST-segment elevation myocardial infarction [STEMI] managed medically or with subsequent percutaneous coronary intervention [PCI] (1.4)

Structure. Enoxaparin sodium is a low molecular weight heparin, LMWH (mean molecular weight of approximately 4,500 Daltons). The drug substance is the sodium salt. The chemical formula is (C26H40N2O36S5)n. The molecular weight distribution is: <2000 Daltons 12 to 20%, 2000 to 8000 Daltons 68 to 82%, and >8000 Daltons≦18%. The systematic (IUPAC) name is 6-[5-acetylamino-4,6-dihydroxy-2-(sulfooxymethyl)tetrahydropyran-3-yl]oxy-3-[5-(6-carboxy-4,5-dihydroxy-3-sulfooxy-tetrahydropyran-2-yl)oxy-6-(hydroxymethyl)-3-sulfoamino-4-sulfooxy-tetrahydropyran-2-yl]oxy-4-hydroxy-5-sulfooxy-tetrahydropyran-2-carboxylicacid.

Production. Enoxaparin sodium is obtained by alkaline depolymerisation of heparin benzyl ester derived from porcine intestinal mucosa. Its structure is characterised by a 4-enopyranose uronate group at the non-reducing end. About 20% (ranging between 15% and 25%) of the enoxaparin structure contains a 1,6 anhydro derivative on the reducing end of the polysaccharide chain.

Mechanism of Action. Enoxaparin binds to and accelerates the activity of antithrombin III. By activating antithrombin III, enoxaparin preferentially potentiates the inhibition of coagulation factors Xa and IIa. The anticoagulant effect of enoxaparin can be directly correlated to its ability to inhibit factor Xa. Factor Xa catalyzes the conversion of prothrombin to thrombin, therefore, the inhibition of this process by enoxaparin results in decreased thrombin levels and ultimately the prevention of fibrin clot formation.

Enoxaparin sodium solution for injection also contains water for injections as an inactive ingredient.

To the best knowledge of the inventor, the use of this compound has not been described in the use of CREVO or BRVO prior to the present invention. The present invention represents a new use for an existing compound.

The present invention comprises the use of enoxaparin sodium or other agent, in a dose and duration sufficient to produce resolution of venous thrombus in the central or branch retinal vein or other structure. One preferred dosing regimen includes 1 mg/kg q12 hours for 3 months. For a typical make adult, this would comprise 80 mg subcutaneously, every 12 hours. This may be given intravenously or other parenteral route or enteral route without departing from the present invention. Furthermore, this or another agent may be given via these routes or via an implanted delivery method, such as via a biodegradable polymer, capsule, microfabricated delivery device, or other method or apparatus.

Alternate heparins, coumarins (including warfarin), antiplatelet agents (including clopidrogel), and other agents may be used without departing from the present invention.

Additional or alternative anticoagulants taught in the present invention may comprise but are not limited to heparin, enoxaparin (including but not limited to brands such as Lovenox), low molecular weight heparin (LMWH), dalteparin sodium (including but not limited to brands such as Fragmin), coumarin, coumadin (including but not limited to brands such as Warfarin), acenocoumarol (including but not limited to brands such as Sintrom and Sinthrome), dicumarol (including but not limited to BisHydroxycoumarin, Bishydroxycoumarin, Dicoumarin, Dicoumarol or other names), phenprocoumon (including but not limited to brands such as Marcoumar, Marcumar and Falithrom), phenindione (including but not limited to brands such as Dindevan), clopidogrel (including but not limited to brands such as Plavix), dabigatran (including but not limited to brands such as Pradaxa, Pradax, and Prazaxa), rivaroxaban (including but not limited to brands such as Xarelto), fondaparinux (including but not limited to brands such as Arixtra), acetylsalicylic acid (including but not limited to brands such as aspirin and USAN), direct factor Xa inhibitor, indirect factor Xa inhibitor, direct thrombin inhibitor, indirect thrombin inhibitor, antithrombin, batroxobin, hementin, or other anticoagulant. Any single, plurality, or combination of these listed or other anticoagulants, derivatives thereof, alternatives thereof, substitutes thereof, or other anticoagulant medications, anticoagulant agents, modalities including but not limited to chemicals and forms of energy for modulation of anticoagulant function may be used without departing from the present invention.

The patient is preferably followed with regular ophthalmologic visits, including slit lamp evaluations of the retina. Furthermore, retinal fluorescein angiograms are performed initially and on occasional follow up visits to assess and document resolution of the thrombus and response to therapy. Other metrics of visual function including but not limited to monitoring at least one of visual symptoms, visual examination, visual function, retinal appearance, retinal vasculature, fluorescein angiogram, visual evoked potential (VEP), electroretinogram (ERG), and other physiological metric of visual function. Any single or plurality of these or other metrics may be used to monitor retinal and visual function.

In one preferred embodiment, monitoring is continued until a desired first therapeutic endpoint is reached. Said therapeutic endpoint may be at least one of a planned duration of therapy and a desired level of visual function. The duration of such therapy may be 24 hours, 48 hours, 72 hours, 1 week, 2 weeks, 2 weeks, 1 months, 2 months, 3 months, 6 months, 9 months, 12 months, 18 months, 24 months, 36 months or other duration. Said desired level of visual function may be stabilization of visual deficit, such as to prevent additional occlusion and visual loss, partial recovery of visual function, or full recovery of visual function. A first anticoagulant may be given until this first therapeutic endpoint is reached, at which time or following which time, therapy may be transitioned to a second anticoagulant. Said transition may be gradual, abrupt, or using a bridge therapy or a gap in therapy. Such therapeutic endpoints include but are not limited to resolution of thrombus, improvement in thrombus, improvement in retinal circulation, improvement in retinal appearance, improvement in metric of visual function, normalization of visual function, normalization of retinal circulation, normalization of metric of visual function, or other metric, symptom, or finding.

Once he thrombus has resolved, it may be appropriate to modify the therapy based upon the individual patient's situation, including but not limited to underlying hematological diagnosis or condition, age, other vascular abnormalities or malformations, prior history of ischemic or hemorrhagic events, future risk of ischemic or hemorrhagic events, profession, hobbies, sports, activities, and other considerations.

In one preferred embodiment, after resolution of the retinal venous thrombus, enoxaparin sodium may be discontinued in favor of an oral agent, such as aspirin, clopidrogel, warfarin, or other anticoagulant, Alternatively, enoxaparin sodium may be continued indefinitely or for another duration, without departing from the present invention. Alternatively, enoxaparin sodium may be discontinued without the subsequent use of another anticoagulant, without departing from the present invention.

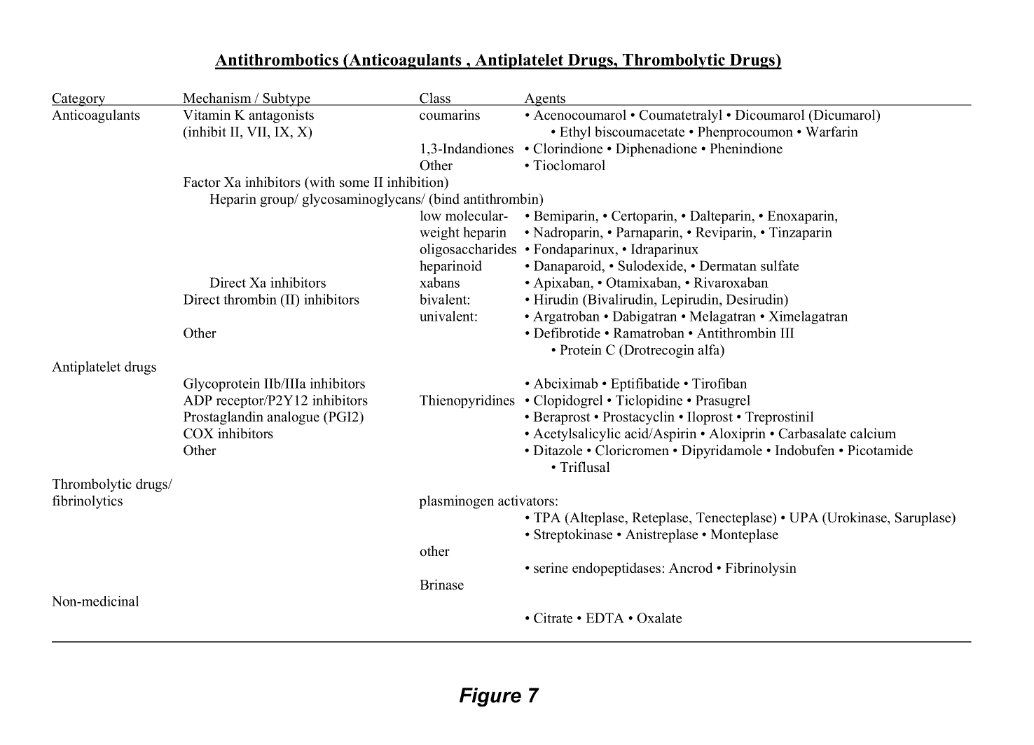

Other pharmaceutical agents may be employed using the methods, apparatus, and methods taught in the present invention. These include but are not limited to other doses, mixtures, and regimens of those agents mentioned. These also include but are not limited to other doses, mixtures, and regimens of, chemical derivatives of, and substitutes for enoxaparin sodium, antiplatelet drugs (including but not limited to Aspirin and other Cyclooxygenase inhibitors, Adenosine diphosphate (ADP) receptor inhibitors (Clopidogrel (Plavix), Ticlopidine (Ticlid)) Phosphodiesterase inhibitors (Cilostazol (Pletal)), Glycoprotein IIB/IIIA inhibitors (Abciximab (ReoPro), Eptifibatide (Integrilin), Tirofiban (Aggrastat), Defibrotide), Adenosine reuptake inhibitors (Dipyridamole (Persantine)), other drug classes and agents, and drugs or agents mentioned in FIG. 7 or comparable, similar, equivalent, or derivative compounds.

### Apparatus for Convenient Injection

Current practice comprises self-administration by a patient, often involving self-injecting in the abdominal subcutaneous adipose tissue twice daily. This can be a disconcerting and at times painful activity. This can negatively affect compliance as well as acceptance of this as a therapeutic option among patients.

The present invention includes a device which automatically advances a thin needle into the subcutaneous tissue, alleviating the patient from the distressing task of advancing a sharp object toward their own body.

In a preferred embodiment, the needle shaft advances along a guide by a fixed travel distance. The guide insures that the needle advances in a direction parallel to its axis, preventing movement perpendicular to its axis, an occurrence readily possible with manual injection and which can result in shear stress in the injected tissue with a tearing effect, and which can induce pain. The mechanism facilitates a wind-up action, such that the user may apply a slow or rapid movement to apply force to the injection trigger device. Once sufficient energy has been stored to perform a successful injection the injection needle rapidly advances along its axis to a prescribed and safe depth into the subcutaneous tissue. This rapid movement, prevents dimpling and deformation of the surface of the skin, an event which can cause pain and discomfort. The rapid movement results in a clean penetration of the needle into the tissue, with less tissue deformation and pain.

A further feature of novelty is an injection system that includes a local anesthetic either provided solely, admixed with another agent to be administered, or included in a binary chamber or multiple chamber device in which an anesthetic is provided as the first or as one of the injections delivered. By this method and with such a device, one can provide injections which are nearly or completely free of pain, thereby improving the patient experience as well as patient compliance, and consequently the effectiveness of treatment. With injections of anticoagulant medications, local hematoma development is common and is a source of injection site bruising and pain. Inclusion of compounds, methods, and apparatus to reduce injection site pain will reduce patient discomfort, improve acceptance and compliance, and thereby improve efficacy of these modalities of therapy.

This injection device, or others which may achieve comparable results using equivalent or different mechanisms or methods may be employed without departing from the present invention. These methods and apparatus may also be used for the delivery of other drugs, compounds, or fluids, without departing form the present invention.

The apparatus and method taught in the parent case is enabling to an engineer or engineering team skilled it the art of developing pharmaceuticals and injection systems, and descriptions of the methods, devices, compounds, and dosing regimens are enabling to a competent pharmaceutical and medical device development team and corresponding clinical practitioners.

### CONCLUSION

It will be appreciated by those skilled in the art that while the invention has been described above in connection with the particular embodiments and examples, the invention is not necessarily so limited, and that numerous other embodiments, examples uses, modifications, and departures from the embodiments, examples, and uses are intended to be encompassed by the claims attached hereto The entire disclosure of each patent and publication cited herein is incorporated by reference, as if each such patent or publication were individually incorporated by reference herein.

I claim:

1. A method for treating a person with central retinal vein occlusion, comprising:
A. Identifying a person with central retinal vein occlusion
B. Administering an anticoagulant

2. The method of claim 1, wherein said anticoagulant is heparin.

3. The method of claim 1, wherein said anticoagulant is enoxaparin.

4. The method of claim 1, wherein said anticoagulant is low molecular weight heparin.

5. The method of claim 5, wherein said anticoagulant is coumadin.

6. The method of claim 1, wherein said anticoagulant is acenocoumarol.

7. The method of claim 1, wherein said anticoagulant is dicumarol.

8. The method of claim 1, wherein said anticoagulant is phenprocoumon.

9. The method of claim 1, wherein said anticoagulant is phenindione.

10. The method of claim 1, wherein said anticoagulant is clopidogrel.

11. The method of claim 1, wherein said anticoagulant comprises at least one of heparin, enoxaparin, low molecular weight heparin, dalteparin sodium, coumarin, coumadin, acenocoumarol, dicumarol, phenprocoumon, phenindione, clopidogrel, dabigatran, rivaroxaban, fondaparinux, acetylsalicylic acid, aspirin, direct factor Xa inhibitor, indirect factor Xa inhibitor, direct thrombin inhibitor, indirect thrombin inhibitor, antithrombin, batroxobin, hementin, other anticoagulant, other modulator of coagulation function, antiplatelet drug, cyclooxygenase inhibitors, adenosine diphosphate receptor inhibitor, phosphodiesterase inhibitor, Glycoprotein IIB/IIIA inhibitor, eptifibatide, Tirofiban, Adenosine reuptake inhibitor, other modulator of coagulation, other modulator of platelet function, other modulator of clot formation, and other modulator of clot breakdown.

12. The method of claim 1, wherein said administering an anticoagulant further comprises intraocular administering an anticoagulant.

13. A method for treating a person with central retinal vein occlusion, comprising administering an anticoagulant to a person with retinal vein occlusion.

14. The method of claim 13, wherein said anticoagulant is a low molecular weight heparin.

15. The method of claim 13, wherein said anticoagulant is enoxaparin.

16. The method of claim 15, wherein the dosing comprises at least 5 mg/kg per day.

17. The method of claim 15, wherein the dosing comprises at least 1 mg/kg per day.

18. The method of claim 15, wherein the dosing comprises at least 0.5 mg/kg per day.

19. The method of claim 15, said administration comprises administration of anticoagulant for a duration of at least one of 24 hours, 3 days, 1 week, 2 weeks, 3 weeks, 3 months, 6 months, 12 months, 24 months, 36 months, or longer than 36 months.

20. A method for treating a person with central retinal vein occlusion, comprising:
A. Identifying a person with central retinal vein occlusion;
B. Administering a first anticoagulant;
C. Monitoring efficacy of first anticoagulant by monitoring at least one of visual symptoms, visual examination, visual function, retinal appearance, retinal vasculature, fluorescein angiogram, visual evoked potential, electroretinogram, and other physiological metric of visual function; and titrating first anticoagulant as necessary.
D. Upon achievement of desired therapeutic endpoint, at least one of tapering or discontinuing said first anticoagulant; and
E. Administering a second anticoagulant.
